(19) United States
(12) Patent Application Publication
(10) Pub. No.: US 2014/0288624 A1
(43) Pub. Date: Sep. 25, 2014

(54) ANATOMY HEATING/COOLING SYSTEM

(71) Applicant: The Plushy Family LLC, Thiensville, WI (US)

(72) Inventors: Laetitia Wasko, Thiensville, WI (US); Antje McClellen, Milwaukee, WI (US)

(73) Assignee: The Plushy Family LLC, Thiensville, WI (US)

(21) Appl. No.: 14/225,325

(22) Filed: Mar. 25, 2014

Related U.S. Application Data
(60) Provisional application No. 61/804,888, filed on Mar. 25, 2013.

(51) Int. Cl. A61F 7/08

(52) U.S. Cl. CPC A61F 7/08; A61F 2007/0007
USPC 607/109

(57) ABSTRACT

A heating/cooling system facilitates heating and/or cooling a person's anatomy. In one implementation, the system comprises a mask, a strap extending from the mask to retain the mask against the anatomy; and at least one pocket behind the mask to receive a heating/cooling pack. In one implementation, the system comprises a soft and flexible head supported portion, a pocket on an inside surface of the head supported portion to be supported against a person's head and a cooling/heating packet removably received within the pocket.

### CROSS-REFERENCE TO RELATED PATENT APPLICATIONS

The present application claims priority from co-pending U.S. Provisional Patent Application Ser. No. 61/804888 filed on Mar. 25, 2013 by Laetitia Wasko and Antje McClellen and entitled PLUSHY MASK COOLING/HEATING SYSTEM, the full disclosure of which is hereby incorporated by reference.

### BACKGROUND

In many circumstances, it may be desirable to heat or to cool a person's anatomy. For example, it may be desirable to cool a forehead of a child who has a fever. It may also be desirable to cool portions of a person's injured anatomy to reduce swelling. It may also be desirable to heat portions of a person's anatomy to heal or loosen muscles. With many individuals, such as children, it is often difficult for the person to continuously hold a heating or cooling pack against his or her anatomy. With many individuals, such as children, it is often difficult to motivate the person to hold a heating or cooling pack against his or her anatomy.

### BRIEF DESCRIPTION OF THE DRAWINGS

FIG. 1 is a front view of an example heating/cooling system.

FIG. 2 is a rear view of the example heating/cooling system of FIG. 1.

FIG. 3 is a top rear perspective view of the heating/cooling system of FIG. 1.

FIG. 4 is a top rear perspective view of an alternative implementation of the heating/cooling system of FIG. 1.

FIG. 5 is a front perspective view of the heating/cooling system of FIG. 1 being worn by a child.

FIG. 6 is a rear perspective view of the heating/cooling system of FIG. 1 being worn by the child.

FIGS. 7A-7C a front views illustrating the heating/cooling system of FIG. 1 being worn along a child's arm.

FIGS. 8A-8C are front views illustrating the heating/cooling system of 1 being worn along a child's leg.

FIG. 9 is a front view of the heating/cooling system of FIG. 1 being worn about a child's midsection.

FIG. 10 is a rear view of the heating/cooling system of FIG. 1 being worn about a child's midsection.

FIG. 11 is a front view of another implementation of the heating/cooling system of FIG. 1.

FIG. 12 is a front view of another implementation of the heating/cooling system of FIG. 1.

FIG. 13 is a front view of another implementation of the heating/cooling system of FIG. 1.

FIG. 14 is a front view of another implementation of the heating/cooling system of FIG. 1.

FIG. 15 is a front view of another implementation of the heating/cooling system of FIG. 1.

### DETAILED DESCRIPTION OF THE EXAMPLE EMBODIMENTS

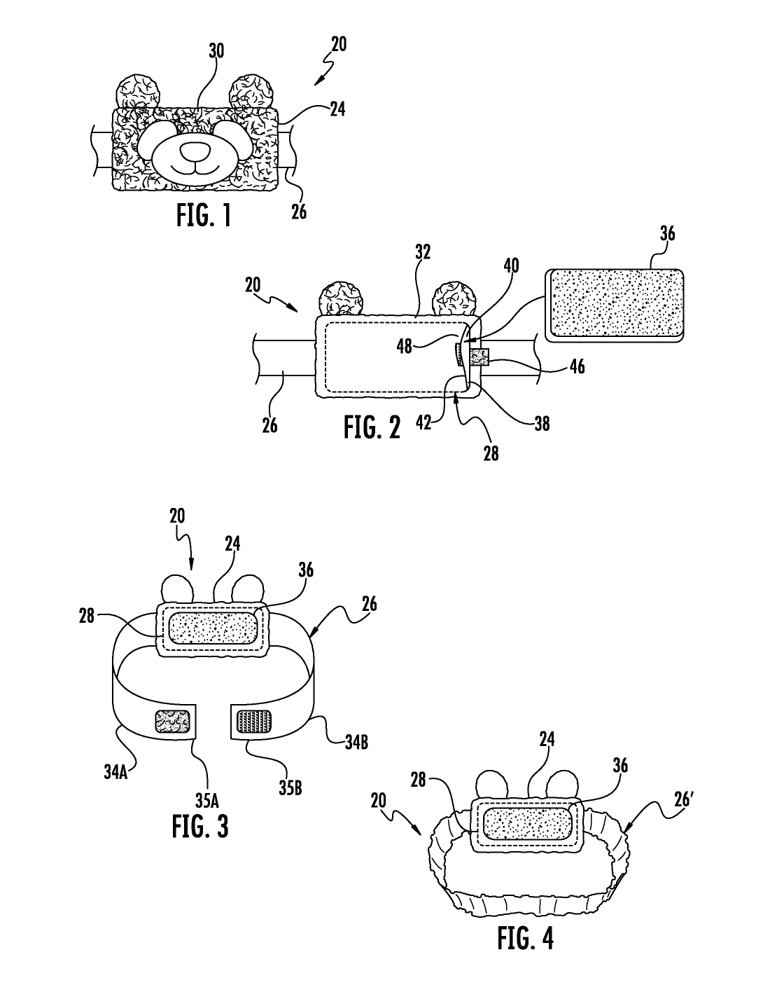

FIGS. 1-4 illustrate an example anatomy heating/cooling system 20. In particular, FIGS. 1-4 illustrate an example mask heating/cooling system. System 20 is configured to retain one or more heating/cooling packs adjacent to or against a person's anatomy. System 20 is further configured to function as a mask, concealing the person's identity for facilitating imaginative play or facilitating self identification by allowing the person wearing the mask to associate himself or herself with a character, animal, sports team, mascot or the like.

System 20 comprises mask 24, strap 26 and one or more pockets 28. Mask 24 comprises one or more structures having printing thereon and/or in the shape of an animal, character, logo, team mascot or the like. In the example illustrated, mask 24 is formed from one or more soft, compressible and/or flexible materials. In one implementation, mask 24 is formed from a soft, compressible and/or flexible fabric. In one implementation, mask 24 is formed from a plush material, similar to those materials found in stuffed animals. In one implementation, the plush material be formed a dense pile of synthetic fibers such as polyester. In yet another implementation, the plush material may be formed from mohair or worsted yarn.

In the example illustrated, mask 24 have a front surface or face 30 (shown in FIG. 1) and a rear surface or face 32 (shown in FIG. 2). In the example illustrated, front face 30 has a three-dimensional shape while rear face 32 is largely two-dimensional, flat or smooth, facilitating conformity to the anatomy to increase the surface area of system 20 having conformal contact with the adjacent anatomy, such as a person's fore head or face. In the example illustrated, mask 24 is formed from a panel of plush material and at least one bulbous panel extending from a front face of the panel to provide mask 24 with its three-dimensional front face 32.

In the illustrated example implementation, mask 20 allows the person wearing the mask to pretend that he or she is an animal or to associate himself or herself with an animal, such as a bear, whether be a brown bear, panda bear or the like. In some implementations, the bear character may also be a cartoon character (for example, from the movie KUNG FU PANDA) or a sports mascot (such as the Chicago Bears). In yet other implementations, front face 30 of mask 24 may comprise other animals, other cartoon characters, other sports mascots, a team logo or other symbol or the like.

As shown by FIG. 3, strap 26 extends from mask 24 and is sized or otherwise configured to wrap about a person's anatomy to secure and hold rear face 32 and the contents of pocket 28 adjacent to the person's anatomy. In the example shown in FIG. 3, strap 26 comprises strap portions 34A, 34B extending from mask 24 and having ends 35A, 35B (collectively referred to as ends 35) releasably securable to one another. In the example illustrated, ends 35 are releasably securable to one another by means of hook and loop (VELCRO) fastening arrangements. In other implementations, ends 35 are releasably securable to one another by a resiliently flexible latching mechanism, by a button, hoax or the like. As shown by FIG. 4, in yet other implementations, system 20 may alternatively comprise strap 26′, wherein strap 26′comprises a single continuous elastic band secured to mask 24 for elastically stretching or about a person's anatomy to support mask 24 and pocket 28 in place against the person's anatomy.

Pocket 28 comprises a pouch or other receptacle sized and shaped to receive a heating/cooling pack 36 (shown in FIG. 2). In one implementation, pack 36 may comprise a pack of ice. In another implementation, pack 36 comprises a pack of a cooling frozen or semi frozen gel or a pack of frozen or semi frozen particles, such as frozen peas. In yet another implementation, pack 36 is configured to emit heat. For example, pack 36 may comprise a pair of ingredients are chemicals which when mixed produce an exothermic reaction, releasing heat (similar to the exothermic packets found in boot warmers or hand warmers).

In the example illustrated, pocket 32 is substantially coextensive with rear face 32 of mask 24. In the example illustrated, pocket 32 is sized to substantially cover a person's forehead, facilitating use of system 20 cool a child or person having a fever. In yet other implementations, pocket 28 may extend along other extents of rear face 32 of mask 24. In yet other implementations, system 20 may include multiple distinct spaced pockets along rear face 32 of mask 24, facilitating selective positioning of one or more appropriately sized and configured heating/cooling packs 36 to selectively heat or cool distinct portions of the anatomy over which mask 24 extends.

In one implementation, pocket 28 is formed from a single panel stitched, bonded, welded or joined to the back face 32 of mask 24 such that the rear face 32 of mask 24 defines a portion of the interior of pocket 28. In yet another implementation, pocket 28 is formed from a sleeve or pocket forming enclosure which is itself attached to rear face 32 of mask 24. In one implementation, pocket 28 forms an interior 38 having a front surface 40 between the interior 38 and mask 24 and a rear surface 42 between the interior 38 and the anatomy against which the system 20 positioned. In one implementation, front surface 40 (or another layer or panel between interior 38 and mask 24 or in front of front surface 40) is formed from a thermally insulative, thermally reflective or radiant barrier material, such as a Poly Therm fleece silver material commercially available from Bosal or TEMPTROL heat reflecting fabric, while rear surface 42 is formed from a porous thermally transmissive material, such as a thin porous fabric. As a result, the heat produced by the pack 36 or the heat absorption by pack 36 (depending upon whether pack 36 is a heating pack or cooling pack) is uni-directionally directed through rear surface 42 towards the anatomy being heated or cooled.

As further shown by FIG. 2, in the example illustrated, pocket 28 further comprises a retainer 46 for closing mouth 48 of pocket 28 to retain pack 36 within pocket 28. In one implementation, retainer 46 comprises a strap releasably securable to rear face 32 of mask 24 utilizing a hook and loop fastening mechanism. In another implementation, retainer 46 comprises a zipper, button or flexible latch. In yet another implementation, retainer 46 comprises an elastic band sewn within or secured about mouth 48, wherein the elastic band stretches during insertion of pack 36 and resiliently returns to a contracted state about pack 36. In yet other implementations, retainer 46 may be omitted.

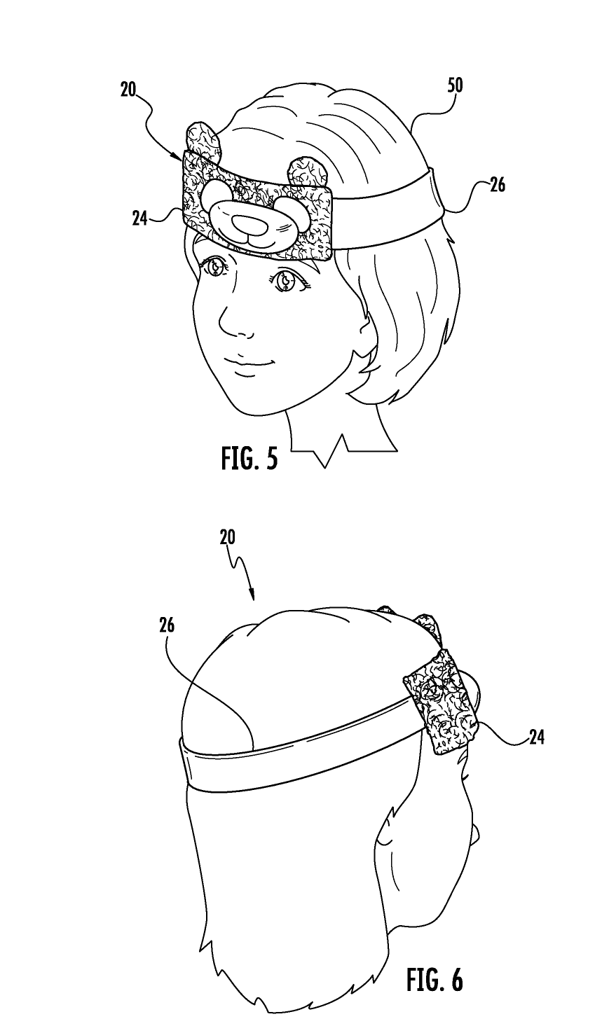

FIGS. 5 and 6 illustrate heating/cooling system 20 being worn about a child's head 50. As illustrated by such figures, strap 26 wraps about head 50 while securing mask 24 across the child's forehead. Pocket 28, containing pack 36, is sandwiched between mask 24 and the person's fore head, applying heat (or absorbing heat). In one implementation, pack 36 comprises an ice pack or a cooler frozen gel pack to absorb heat from the person's fore head, such as when the child has a fever. Because mask 24 conceals or covers pack 36, which is behind mask 24, the child may be less likely to focus his or her attention upon the pack 36 being held against his or her for head. Moreover, because mask 24 also functions as a mask allowing the child to pretend that he or she is something else or allowing the child to associate himself or herself with the team, logo, mascot, character animal of mask 24, the child may be more motivated, more willing or less resistant to continue to wear mask 24 and heating/cooling system 20.

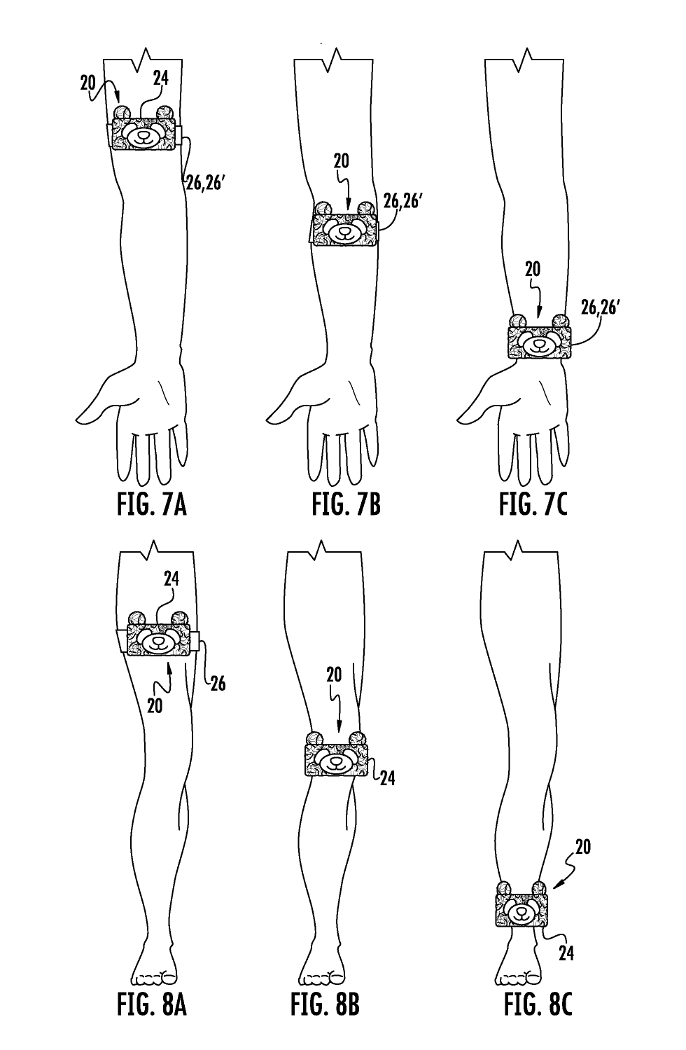

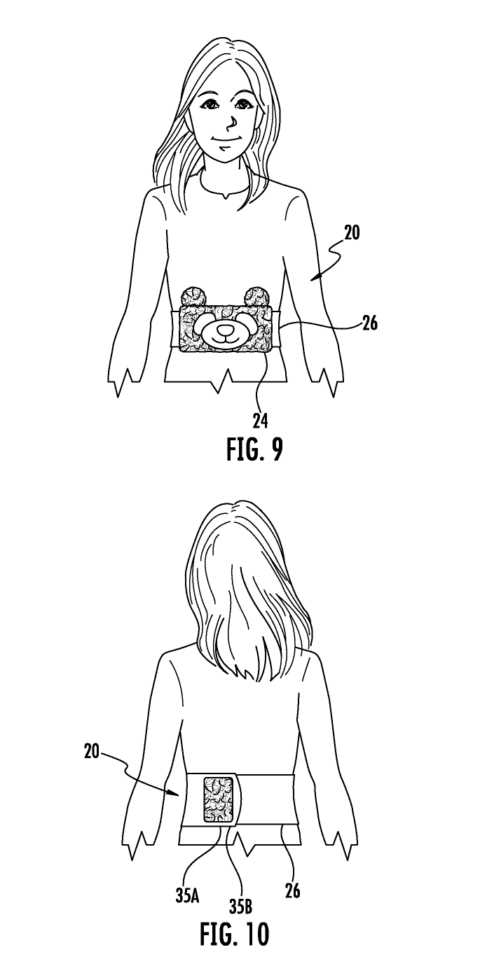

In the example shown in FIGS. 1-6, heating/cooling system 20 is illustrated as being severely configured for being supported on a person's head with mask 24 extending across the person's fore head. In other implementations, heating/cooling system 20 is adaptable to support a heating or cooling pack 36 adjacent to other portions of a person's anatomy. Such adjustment may involve reshaping a resizing of mask 24, strap 26, pocket 28 and the heating/cooling pack 36. For example, as shown by FIGS. 7A, 7B and 7C strap 26, 26′may be resized or reconfigured to secure mask 24 against narrower portions of a person's anatomy such as about a person's upper arm, elbow, forearm or wrist. As shown by FIGS. 8A, 8B and 8C, system 20 may be reconfigured and resized to secure mask 24 against or about a person's thigh, knee, lower leg or ankle As shown by FIGS. 9 and 10, system 20 may be reconfigured a resized to wrap about a person's chest, abdomen or waist.

FIG. 11 illustrates an example heating/cooling system 120, another implementation of system 20. System 120 is similar to system 20 except that system 120 comprises mask 124 and pockets 128A, 128B and 128C (collectively referred to as pockets 128). Mask 124 similar to mask 24 except that mask 124 is configured to overlie additional portions of a person's face. As such, mask 124 comprises eye openings 160, nose openings 162 and mouth opening 164. In lieu of being formed from a plush material, mask 124 is formed from a polymer or other flexible or soft material. Because mask 124 covers substantially and entirety of a person's or child's face, mask 124 may better facilitate imaginative play or use of mask 124 by the person or child. At the same time, mask 124 provides a greater area for supporting and concealing a greater number of pockets or larger pockets 128, allowing mask 124 to provide pockets opposite to additional portions of the person's face, beyond just the person's fore head.

Pockets 128 are similar to pocket 28 in that pockets 128 comprise pouches, chambers, cavities are like to removably receive heating/cooling packets or packs 36. In one implementation, the front side of each of pockets 128 is aligned with a thermally insulative or reflective layer or panel affirmed described above) while the rear panel or side of each of pockets 128 (closest to the person's face) is formed from a thermally transmissive or thermally conductive material such as a thermally transmissive or thin fabric. As noted above, in some implementations, pockets 128 may additionally include a retainer 46.

In the example illustrated, pocket 128A is similar to pocket 28 in that pocket 128A is substantially coextensive with a person's fore head when mask 124 is being worn. Pockets 128B and 128C extend behind mask 124 below eye openings 160. Pockets 128B and 128C are located so as to extend substantially opposite to or across from a person's cheekbones. As a result, pockets 128B and 128C retain and hold properly sized cooling packets 36 when the person has a bruise or other injury to the person cheekbones. Because system 120 comprises multiple pockets 128 at different locations, a person may selectively fill such pockets with heating/cooling packets or packs as needed depending upon what portions of the person's anatomy or what portions of the person's face require attention through cooling, such as with a bruise.

FIG. 12 illustrates heating/cooling system 220, another example implementation of system 20. System 220 is similar to system 120 except that system 220 comprises mask 224 in lieu of mask 124 and further comprises pocket 228 carrying a corresponding cooling pack 36. Mask 224 is similar to mask 124 except that mask 224 comprises a printed or otherwise opaque eye covering portion 240 in lieu of one of eye openings 160. Eye covering portion 240 extends across or over a person's eye when mask 224 is worn. Eye covering portion 240 may include a shaped, painted or printed upon eye. Eye covering portion 240 extends over and in front of pocket 228.

Pocket 228 is similar to pockets 28 and 128 in that pocket 228 is shaped and sized to hold and retain a heating/cooling pack, such as cooling pack 236. In one implementation, pocket 228 has a panel or surface between the interior of pocket 228 and the front of mask 224 which is thermally reflective or thermally insulative and has another panel or surface between the interior of pocket 228 and the person's anatomy that is thermally conductive or thermally transmissive. Pocket 228 facilitates the retention of cooling pack 236 across and about a person's eye. Pocket 228 facilitates cooling of the eyebrow or tissue about the person's eye which may be bruised. At the same time, mask 224 conceals the black eye or bruised eye as it is being treated.

FIG. 13 illustrates heating/cooling system 320, another example implementation of heating/cooling system 20. Heating/cooling system 320 is similar to heating/cooling system 220 except that system 320 comprises mask 324, pockets 328A, 328B (collectively referred to as pockets 328) and cooling pack 336. Mask 324 is similar to mask 124 except that mask 324 does not extend over a person's mouth or nose and that mask 324 supports additionally sports pockets 328A and 328B behind eye openings 160.

Pockets 328 are similar to pocket 228 except that pockets 328 comprise openings 370 in alignment with eye openings 160 in mask 324. Cooling pack 336 is similar to cooling pack 236 exhibit cooling pack 336 comprises an annular ring or other member having a central opening 372 which extends into alignment with either hoping 370 and the eye-opening 160 when resting are supported within the associated pocket 328. System 320 allows a cold pack, such as cold pack 336, to be positioned about the eye of the person wearing mask 324, such as when the person or child has a bruise about his or her eye. At the same time, vision is not blocked.

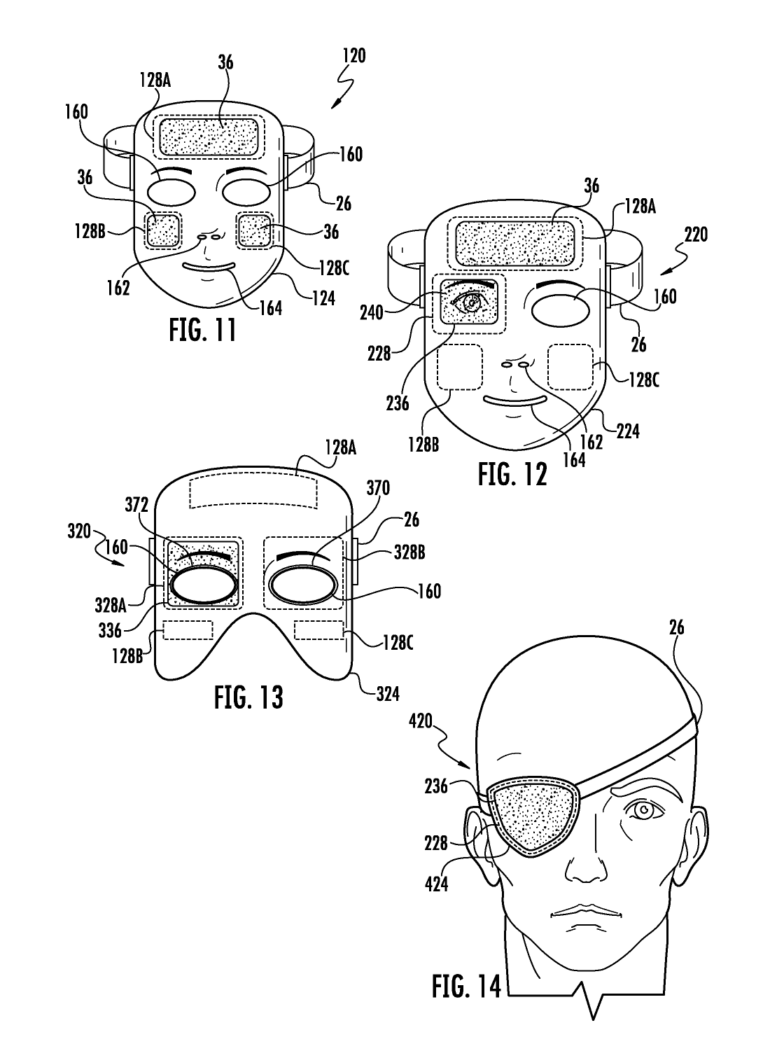

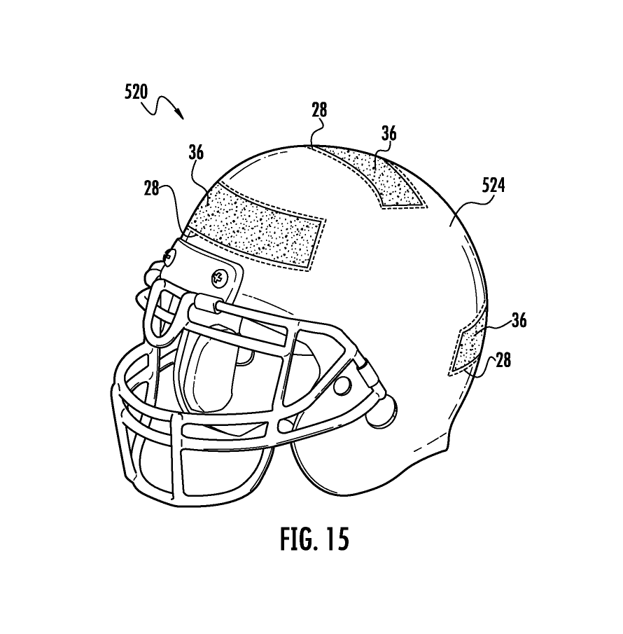

FIG. 14 illustrates heating/cooling system 420, another implementation of heating/cooling system 20. System 420 is similar to system 220 except that some 420 comprises mask 424. Mask 424 is configured as an “eye patch” to cover one I substantially only cover one eye. In one implementation, mask 424 figure similar to a pirate's eyepatch. Mask 424 comprises pocket 228 that removably receives cooling pack 36. In yet other implementations, mask 424 and pocket 228 may include aligned openings similar to system 320 to contain an annular cooling pack 336 to allow the eye patch to be worn without blocking vision.

FIG. 15 illustrates heating/cooling system 520, another implementation of heating/cooling system 20. System 520 is similar to system 20 except the system 520 comprises head support 524 and omits straps 26. Head support 524 comprises a hat, helmet or the like made of soft, depressable lightweight fabric or other material. In the example illustrated, head support 524 is in the shape of a football helmet, wherein as a support 524 includes internal pockets 28 to receive heating/cooling packs 36 at one or more selected locations within or along the interior of head support 524. System 520 facilitates support of cooling packs 28 adjacent to rear, top inside portions of a child's head (in addition to possibly the child's forehead), such as when child has a ball or bruise on his or her head. Although illustrated as a football helmet, head support 524 may alternatively comprise a baseball hat or cap, a fireman's helmet, a police hat or any other form of headwear.

Although the present disclosure has been described with reference to example embodiments, workers skilled in the art will recognize that changes may be made in form and detail without departing from the spirit and scope of the claimed subject matter. For example, although different example embodiments may have been described as including one or more features providing one or more benefits, it is contemplated that the described features may be interchanged with one another or alternatively be combined with one another in the described example embodiments or in other alternative embodiments. Because the technology of the present disclosure is relatively complex, not all changes in the technology are foreseeable. The present disclosure described with reference to the example embodiments and set forth in the following claims is manifestly intended to be as broad as possible. For example, unless specifically otherwise noted, the claims reciting a single particular element also encompass a plurality of such particular elements.

What is claimed is:

1. An apparatus comprising:
a mask;
a strap extending from the mask to retain the mask against an anatomy; and
at least one pocket behind the mask to receive a heating/cooling pack.

2. The apparatus of claim 1, wherein the mask is sized to cover a forehead.

3. The apparatus of claim 2, wherein the mask is formed from a plush.

4. The apparatus of claim 2, wherein the mask comprises a three-dimensional front face in a two-dimensional rear face forming an interior of the at least one pocket.

5. The apparatus of claim 2, wherein the mask comprises a first eye opening and a second eye opening, the first eye-opening and the second eye-opening corresponding to first and second eyes of a person wearing the mask.

6. The apparatus of claim 3, wherein the at least one pocket is coextensive with and extends about the first eye-opening.

7. The apparatus of claim 1, wherein the mask comprises a first eye opening, the first eye-opening corresponding to a first eye of a person wearing the mask and wherein the at least one pocket extends around the first eye-opening but does not cover a forehead of the person.

8. The apparatus of claim 1, wherein the mask comprises a first eye opening and a second eye opening, the first eye-opening and the second eye-opening corresponding to first and second eyes of a person wearing the mask and wherein the at least one pocket extends above and along the first eye-opening.

9. The apparatus of claim 1, wherein the mask comprises an eye-opening and an eye covering portion, the eye-opening correspond to a first eye of a person wearing the mask in the eye covering portion corresponding to a second eye of the person wearing the mask, wherein the at least one pocket extends behind the eye covering portion.

10. The apparatus of claim 1, wherein the mask comprises an eye covering portion and wherein the at least one pocket extends behind the eye covering portion.

11. The apparatus of claim 1, wherein the at least one pocket comprises a plurality of pockets to independently receive different heating/cooling packs.

12. The apparatus of claim 11, wherein the plurality of pockets comprise a first pocket having a first size and a second pocket having a second size greater than the first size.

13. The apparatus of claim 1 further comprising the heating/cooling pack.

14. The apparatus of claim 13, wherein the heating/cooling pack comprises an annular ring.

15. The apparatus of claim 1, wherein the mask comprises:
a panel of plush material, the panel having a front face and a rear face, wherein the rear face defined an interior of the at least one pocket; and
at least one bulbous panel extending from the front face.

16. The apparatus of claim 1, wherein the strap comprises an elastomeric strap to encircle the anatomy.

17. The apparatus of claim 1, wherein the strap comprises a first portion and a second portion, the first portion and the second portion being releasably secured to one another.
The apparatus of claim 1, wherein the mask has a shape selected from a group of shapes consisting of: an animal's head, a cartoon character and a sports mascot.

18. The apparatus of claim 1, wherein the mask comprise a head covering portion.

19. The apparatus of claim 1 further comprising:
a front panel between an interior of the pocket and a front of the mask, the front panel formed from a first material, the first material being selected from a group of materials consisting of: a thermally reflective material and a thermally insulative material; and
a rear panel between an interior of the pocket and a rear surface of the pocket, the rear panel formed from a second material different than the first material, the second material beings selected from a group of materials consisting of: a thermally conductive material and a thermally transmissive material.

20. A heating/cooling system comprising:
a soft and flexible head supported portion;
a pocket on an inside surface of the head supported portion to be supported against a person's head; and
a cooling/heating packet removably received within the pocket.
